(19) United States
(12) Patent Application Publication
(10) Pub. No.: US 2009/0047334 A1
(43) Pub. Date: Feb. 19, 2009

(54) TRANSDERMAL PATCH FOR EXTENDED DELIVERY OF CALCIUM

(76) Inventor: Patricia Williams, Raleigh, NC (US)

Correspondence Address: PASSE' INTELLECTUAL PROPERTY, LLC, 1717 BRASSFIELD RD., RALEIGH, NC, 27614, US

(21) Appl. No.: 12/190,405

(22) Filed: Aug. 12, 2008

Related U.S. Application Data
(60) Provisional application No. 60/955,488, filed on Aug. 13, 2007.

(51) Int. Cl. A61K 9/70; A61K 33/00

(52) U.S. Cl. 424/449; 424/722

(57) ABSTRACT

The present invention relates to a method for delivery of calcium that avoids the problems with orally given calcium tablets. Particularly, the present invention is a transdermal patch for providing between 40 mg and 500 mg of bioavailable calcium to a patient.

This application claims priority of U.S. application Ser. No. 60/955,488 filed on Aug. 13, 2007 and incorporated herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a formulation and delivery method for administration of elemental calcium to the human body. In particular the present invention relates to the delivery of calcium using a transdermal patch in order to deliver at least a one day supply of calcium.

2. Description of Related Art

Calcium is an essential mineral nutrient for the human body. Approximately 99% of the calcium in the body is deposited in the bones and teeth with the remaining calcium present in body fluids in either a bound state (e.g. to albumin and globulins) or in a free ionic state. Ionic calcium (Ca++) is the physiologically active form of this mineral in the body. Ionic calcium exists in the blood as described above but also exists in the bone and teeth and movement of calcium to and from the blood stream and the teeth and bones occurs constantly. A proper balance of calcium is necessary for strong bones and teeth and an imbalance in this process can lead to osteoporosis, brittle teeth and bones, skeletal muscle spasms, abnormal heartbeat and the like.

The movement of calcium in the body depends largely on the available ionic calcium and the particular needs of the individual. Individual needs range from those of a new born to differences between men and women, disease states, and certain conditions such as spinal cord injuries. Accordingly the minimum daily requirement for an individual can vary greatly. In general in the United States minimum daily requirements of calcium range from about 400 to about 1500 mg per day based on oral supplementation.

The available body calcium is normally derived from dietary intake and insufficient dietary intake is often the cause of insufficient calcium. In addition to dietary intake factors which affect absorption of calcium, an imbalance or problem with the bodies regulating mechanisms can also affect available ionic calcium. Low PTH is often a cause of calcium problems and is usually accommodated with shots of PTH. Vitamin D affects the rate of absorption of calcium from the gut. Not enough Vitamin D and there is not enough absorption of calcium. Too much Vitamin D and an individual can suffer from calcium toxicity (appetite loss, nausea, vomiting, abdominal pain, confusion, seizures and coma). Vitamin D supplementation is often prescribed along with calcium supplementation but frequently Vitamin D levels are never checked and the amount of Vitamin D administered is a guess at best. Even further too much Vitamin D can lead to tissue accumulation, and thus overdoses of this fat soluble vitamin.

Calcium is available from a wide range of foods from dairy sources, and oral supplementation of calcium is widely prescribed. However, because of the wide variation in conditions for absorption of elemental calcium from these sources the amount of calcium actually absorbed from ingested sources varies extremely widely. Absorption from the commonly available calcium salts (carbonate, citrate, phosphate, gluconate, glubinate and lactate) vary widely but in general it is assumed that only about 30% of the available elemental calcium is actually absorbed and bio available from this method of administration. In some cases the oral administration of calcium results in 10% or less of the available calcium being absorbed through the stomach into the blood stream.

In addition to general poor absorption, the bioavailability decreases with digestive problems. For example, if an individual is not producing sufficient hydrochloric acid, the bioavailability of calcium can drop significantly from the already low amounts from oral supplementation.

A further problem with oral calcium supplementation is that preparations have a tendency to cause stomach upset. Such problems can be mitigated by ingestion of food; however, ingestion of food can itself affect the rate of absorption of calcium, and a prolonged absorption time can mean calcium leaves the stomach before maximum absorption can occur. Some problems can be avoided by administering calcium twice a day; however, the problems of forgetting a dosage increase greatly with patients who have to take calcium twice a day.

Yet another problem with calcium supplementation is the fact that in order to get the high dosages into the blood stream, tablets tend to be in the 500 to 600 mg range, the tablets are excessively big and many individuals find it extremely difficult if not impossible to ingest even a single tablet.

Intravenous (IV) calcium supplementation is often prescribed for the most serious of calcium intake problems. An IV supplement can avoid many of the problems with oral administration even when some absorption factors are low or missing. It is clear however that daily shots are not only extraordinarily expensive, they also have a low compliance rate and can easily lead to an overdose situation.

Accordingly, there is a clear need for a means to deliver calcium which avoids as many of the above administration problems of calcium as possible and still has high compliance as well as a decrease in the side effects and number of administrations necessary.

One attempt to administer calcium in a different manner is a product currently available from Panacea Products called Seablue. Seablue is a dermal vitamin cream comprising magnesium and various vitamins Bs in a gel carrier. The product without the calcium was first described in US patent application 2005/0196433 to Brierre published Sep. 8, 2005 to which calcium was subsequently added but not described in the application. The composition contains among the other vitamins and minerals 37.5 mg of calcium; however, it is not clear if this is elemental calcium. The current web site www seabluevitamins com states that the product must be used 2 or 3 times a day but no equivalence to 1200 mg or 1500 mg or any equivalence to any amount of oral calcium is stated. It appears though that they are using 90% absorption with about 37.5 mg equivalent to about 400 mg orally. This product appears to have some improvements to the other current methods of administering calcium; however, the cream still has some serious drawbacks. First the cream needs to be applied several times a day, and second, when applying by rubbing it in it is possible to apply varying amounts each time you use the product. In additions methods of rubbing and the time the product is left on vary greatly with a cream designed to be used to deliver calcium. Further Administration of other minerals and vitamins competes with the calcium and further causes absorption problems. Lastly, it does not appear that the present formulation would work in a patch formulation or that any type of transdermal patch would work for calcium

### SUMMARY OF THE INVENTION

It has been discovered that by administration of the formulation and method of the present invention that calcium can be delivered in a lower dosage of elemental calcium than orally and administer without the problems of either oral or IV administer calcium. Further, that a full day's amount of calcium can be administered and administration can be done in a measured amount without the need to learn any application techniques.

Accordingly the present invention relates to a transdermal patch for the delivery of dietary supplemental calcium to an individual in need thereof comprising:
a) one or more release layers comprising an absorbable calcium and a release membrane designed to release calcium to the skin of the individual;
b) a support layer comprising a backing material and an area of adhesive to apply the patch to the skin.

These and other objects of the present invention will be clear when taken in view of the detailed specification and disclosure in conjunction with the appended figures.

### BRIEF DESCRIPTION OF THE DRAWINGS

FIG. 1a is a side view of a transdermal patch having a single release layer

FIG. 1b is a side view of a transdermal patch having 2 release layers on top on one another.

FIG. 1c is a side view of a transdermal patch having 2 side by side release layers

### DETAILED DESCRIPTION OF THE INVENTION

It has heretofore been believed that the only way to treat a calcium deficiency was either through oral or injectable calcium supplements. However, oral supplements are subject to an extremely wide variation in absorption rates due to the salt used, stomach condition of the individual, and the availability of absorption aids. They are also difficult to take and must be spread out over a period of time to minimize the oral side effect of calcium. Also, injectable calcium must be administered by a medical professional and can have serious side effects and even lead to death. Recently, a dermal cream containing calcium has been introduced; however, it is not formulated nor is it clear it can be formulated to provide a daily dosage in one use. Further the product clearly can have wide variations of delivered calcium depending on how the cream is measured and applied. In spite of the long standing need for a safe and effective method for administering elemental calcium no other methods of administering calcium by itself have been used.

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one as or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

As used herein the term “transdermal patch” refers to a medicated adhesive patch that is placed on the skin to deliver a time released dose of medication (calcium) through the skin and into the bloodstream. Typically prescription pharmaceuticals are the choice for use with this type of delivery system with the first patch delivering select dosages of nicotine. While a normally higher dosage needs to be delivered than normally used with these types of delivery systems, it has surprisingly been found that large quantities of calcium can be delivered.

As used herein “dietary supplement calcium” refers to calcium that when delivered to the blood stream can form an ionic form of calcium. As described above, the ionic form of calcium is the form that is bioavailable in the bloodstream for the individual's dietary calcium needs. The calcium can be delivered in a pharmaceutically acceptable salt of calcium. So, for example, the carbonate or gluconate salt could be used as the form of calcium used in the present invention. While most any salt could be used in one embodiment, the calcium salt is selected from the calcium salts approved for oral ingestion.

As used herein the phrase “absorbable calcium” refers to formulating the selected salt of calcium into a form that can be absorbed through the skin. Because there are relatively large amounts of calcium needed to cross the skin barrier, the calcium would be formulated into either a suspension such as a slurry or in one embodiment a matrix of ingredients. Most calcium salts are poorly soluble in water and completely insoluble in the more common alcohol based agents used to suspend and cause absorption of a selected composition. Accordingly, a pharmaceutically acceptable slurry or matrix is formed using a suspension agent for this type of release and solubility. The absorbable calcium would then be released from the patch at a rate compatible with the absorption rate of the formulation used to formulate the calcium selected.

As used herein the phrase “one or more release layers” refers to formulating the desired amount of calcium into a layer which will diffuse over a period of time. While one reservoir type layer could be used, one embodiment of this invention provides enough calcium to provide a day's worth of calcium in a single patch. One embodiment would be to have a plurality of such layers. These layers could be side by side or on top of one another with property of each layer releasing at a different rate. Because there is relatively high bioavailability of calcium via the patch route, one can use as little as 10% of the normal oral dose and still achieve a minimum daily requirement of calcium in the bloodstream. Accordingly, one embodiment of a patch provides between 40 mg and 500 mg of bioavailable calcium, and in one embodiment the amount is from about 40 mg to about 150 mg of calcium. Yet another embodiment provides from about 80 mg to about 150 mg of calcium. In these embodiments the amount of calcium could be in one layer or could be divided equally or non-equally in a plurality of layers. One embodiment of the invention is two side-by-side reservoirs have roughly equal amounts of calcium in each.

As used herein the term “release membrane” refers to a membrane in the calcium layer that controls release of the calcium from the layer and delivers the calcium to the skin. A membrane's ? that is compatible with the suspension or matrix of the present invention is used. One such compatible membrane is the ethylene vinyl acetate membranes. These are best with a reservoir type layer and surprisingly useful help with the large quantity of calcium present in the patch. The caliper of each membrane can be selected for the release time and amount of calcium to be released. The release membrane can be chosen to release a wide variation of times. In one embodiment the patch releases the calcium over a period of at least 3 hours. In yet another embodiment, it releases the calcium over a period of at least 24 hours. In yet

The support layer of the present invention comprises a backing material and an adhesive designed to adhere to the patch to the skin. The backing material can be selected from polymeric materials. In one embodiment the polymeric material is water resistant or water impermeable. The backing material can be colored a skin color or any other color desired for the patch. The backing material is chosen larger than the calcium layer or layers. In this way the portion that is larger than the calcium layer and facing the skin when applied is covered with an adhesive. The adhesive is a skin compatible adhesive. Such adhesives are well known in the art and selectable by someone in the art. It is necessary though to provide enough adhesive to stick the large patch of the present invention, and one skilled in the art will be taught in an embodiment herein to select a larger surface area than normally chosen for patches.

In one embodiment of the invention the present invention only provides calcium as the active ingredient of the patch. In this embodiment no other vitamin, mineral or other pharmaceutical preparation is included thus providing a larger amount of calcium release in the patch. In other words, the patch would consist essentially of only a) and b) as described above.

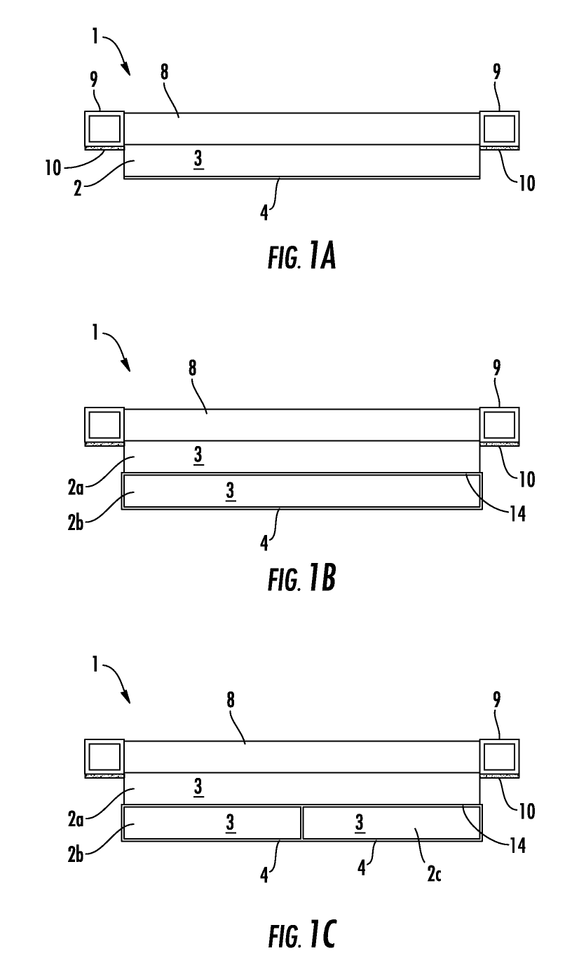

Now referring to the drawings, FIG. 1a is a side view of an embodiment of the present invention where there is a single release layer. The transdermal patch 1 consists of release layer 2 containing absorbable calcium 3. The calcium 3 is released by the membrane 4, which is on what is considered the skin side of the transdermal patch 1. The backing layer 8 also consists of the area larger than the release layer 2. Also shown on the area larger than the release area 9 is the adhesive 10 for sticking the transdermal patch 1 to an individual's skin.

FIG. 1b is a side view of an embodiment of the invention where there are two release layers positioned one on top of the other. In the view of FIG. 1b, the transdermal patch 1 is shown to have release layers 2a and 2b. They are positioned on top of one another. Layer 2a and 2b each contain an absorbable calcium 3. In this embodiment each of the layers has a membrane 4 and 14 for controlling release. In this embodiment layer 2a releases onto layer 2b and must pass through this layer before reaching the skin. Layer 2a can have the same or different release characteristic but the difference in travel distances makes the release different for each layer even if the calcium release properties are the same for both layers 2a and 2b.

In FIG. 1c we have a side view where there are three release layers. In this embodiment the transdermal patch 1 has both side by side layers and on top of one another layers. Layer 2a is shown as in FIG. 1b. It is a complete layer and must pass through either layer 2b or 2c before reaching the skin. Layers 2b and 2c are side by side relative to one another. They are shown as relatively smaller than layer 2a. The release membranes 4 can be the same or different, and likewise, the release characteristics of the calcium 3 can be the same or different. In this embodiment though one embodiment of selecting the layers would be to have each of the 3 layers release at different rates by varying either the calcium release rate or the membrane or both such that there is essentially continuous release over a desired period of time, for example, at least 24 hours or greater than 48 hours.

The above description and example are for the purpose of teaching the person of ordinary skill in the art how to practice the present invention, and it is not intended to detail all those obvious modifications and variations of it which become apparent to the skilled worker upon reading the present invention description. It is intended, however, that all such modifications and variations be included within the scope of the present invention, which is defined by the following claims. The claims are intended to cover the claimed steps and components, in any sequence, consistent with the present invention that is effective to meet the objectives herein intended unless the context specifically indicates to the contrary.

What is claimed is:

1. A transdermal patch for the delivery of dietary supplemental calcium to an individual in need thereof comprising:
a) one or more release layers comprising an absorbable calcium and a release membrane designed to release calcium to the skin of the individual;
b) a support layer comprising a backing material and an area of adhesive to adhere the patch to the skin.

2. A transdermal patch according to claim 1 wherein the patch provides between about 40 and 500 mg of bioavailable calcium.

3. A transdermal patch according to claim 2 wherein the patch provides 40 to 150 mg of calcium.

4. A transdermal patch according to claim 1 wherein there are a plurality of layers and wherein each layer releases calcium at a different rate to the individual's skin.

5. A transdermal patch according to claim 1 or claim 3 which provides calcium release over a period of at least 24 hours.

6. A transdermal patch according to claim 1 wherein the calcium is released to the skin over a period of at least 3 hours.

7. A transdermal patch according to claim 1 which consists essentially of a) and b).

8. A transdermal patch according to claim 1 wherein the calcium is prepared in a formulation selected from the group consisting of solution, suspension and matrix.

9. A transdermal patch according to claim 1 wherein the release membrane is an ethylene vinyl acetate.

10. A transdermal patch according to claim 1 wherein the release layer is a reservoir type layer.

11. A transdermal patch according to claim 1 wherein the release layer is a matrix type layer.
